(19) United States
(12) Patent Application Publication
(10) Pub. No.: US 2014/0315224 A1
(43) Pub. Date: Oct. 23, 2014

(54) DETECTION DEVICE, DETECTION STRIP, AND DETECTION SYSTEM

(71) Applicant: National Tsing Hua University, Hsinchu city (TW)

(72) Inventors: Min-Yen HSU, Hsinchu city (TW); Chung-Yao YANG, Hsinchu city (TW); Chao-Min CHENG, Hsinchu city (TW)

(21) Appl. No.: 14/319,726

(22) Filed: Jun. 30, 2014

Related U.S. Application Data
(63) Continuation-in-part of application No. 13/867,409, filed on Apr. 22, 2013.

(30) Foreign Application Priority Data
Jan. 8, 2013 (TW) 102100580

(51) Int. Cl. G01N 33/543

(52) U.S. Cl. CPC G01N 33/54306
USPC 435/7.92; 435/287.2

(57) ABSTRACT

A detection device applied for detecting body fluids includes a substrate and a plurality of anti-growth factor antibodies. The substrate includes at least one reaction portion. The reaction portion includes a fiber-based material. Anti-growth factor antibodies are disposed on the fiber-based material. The present invention further provides a detection strip and a detection system for detecting body fluids. The present invention is advantageous for easy operation, lower amount of reagents and rapid analysis.

### CROSS REFERENCE TO RELATED APPLICATIONS

This application is a Continuation-In-Part of U.S. application Ser. No. 13/867,409 filed on Apr. 22, 2013, which claims priority under 35 U.S.C. §119(a) on Patent Application No(s). 102100580 filed in Taiwan, Republic of China on Jan. 8, 2013, the entire contents of which are hereby incorporated by reference.

### BACKGROUND OF THE INVENTION

1. Field of Invention

The present invention relates to a detection device, a detection strip, and a detection system, and more particularly, to a detection device, a detection strip, and a detection system applied for detecting eye vascular lesion.

2. Related Art

With the development of technology and biotechnology medical care, the average human lifespan has significantly extended. With the changing of lifestyle, the number of patients suffering from diabetes and macular degeneration also increases. These two diseases are proved to be highly relevant to the concentration of vascular endothelial growth factor. When the concentration of VEGF increases, lesions of diabetic retinopathy or macular degeneration become worse and may even lead to blindness.

In order to cope with these diseases, at least two types of antibody drugs are developed against VEGF. One is Bevacizumab (Avastin), and another one is ranibizumab (lucentis), wherein the latter one is provided with less molecular weight. Both of these drugs are used to reduce the concentration of VEGF in eyes so as to reduce vascular proliferation and prevent deterioration in vision.

However, due to complexity and specialization of the nervous structure of the eyes, general blood testing is not applicable to assess lesion activity of eye diseases. Besides, there is no suitable method to sample eyes fluid for analysis of the growth factors in lesions. Although these two drugs can be applied for the disease, the molecular detection devices applied for eye disease are still poor. Hence, the concentration of angiogenesis in eye cannot be effectively detected or tracked, not to mention adequately assess the amount of drug and mode of administration facilities.

In recent years, researchers have tried to use traditional ELISA assay to detect antibody or antigen, thus executing the detection of VEGF and monitoring of disease activity. The result will help clinicians determine the dose of immunosuppressive drugs.

However, the detection of traditional method needs to obtain detection sample by drawing blood of patients. Otherwise, relatively large number of reagents is required, and longer time is required for detection, which virtually extending the time for diagnosing and increasing the burden on the patient. The detection equipments of traditional ELISA require high cost. Furthermore, traditional ELISA needs to be processed in the laboratory environment with specific apparatus, which is unfavorable for clinical detection.

Therefore, it is an important subject to provide a detection device obtaining the advantages of easy and rapid operation in order to improve clinical applicability.

### SUMMARY OF THE INVENTION

In view of the foregoing, it is an object of the present invention to provide a detection device obtaining the advantages of easy and rapid operation in order to improve clinical applicability.

To achieve the above, the present invention discloses a detection device applied for detecting body fluids comprising a substrate and a plurality of anti-growth factor antibodies. The substrate includes at least one reaction portion with a fiber-based material. Anti-growth factor antibodies are disposed on the fiber-based material.

In one embodiment of the present invention, the body fluid sample is an aqueous humor and vitreous humor.

In one embodiment of the present invention, the substrate includes at least one non-reaction portion, and the non-reaction portion is covered by hydrophobic material.

In one embodiment of the present invention, the substrate includes two reaction portions, and the reaction portions are separated by the non-reaction portion.

In one embodiment of the present invention, the fiber-based material is high density fiber-based material with an average pore size ranged from 0.7 to 12 micrometers.

In one embodiment of the present invention, the anti-growth factor antibodies are anti-angiogenic factor antibody.

To achieve the above, the present invention discloses a detection strip applied for detecting a body fluid sample comprising a sampling portion, a transferring portion and a reaction portion. The transferring portion is disposed between the sampling portion and the reaction portion, the reaction portion includes a fiber-based material, and a plurality of anti-growth factor antibodies are disposed on the fiber-based material.

In one embodiment of the present invention, the body fluid sample is an aqueous humor and vitreous humor.

In one embodiment of the present invention, the fiber-based material is high density fiber-based material with an average pore size ranged from 0.7 to 12 micrometers.

In one embodiment of the present invention, the anti-growth factor antibodies are anti-angiogenic factor antibody.

To achieve the above, the present invention discloses a detection system applied for detecting a body fluid sample of an organism comprising a detection strip comprising a sampling portion, a transferring portion and a reaction portion. The transferring portion is disposed between the sampling portion and the reaction portion, the reaction portion includes a fiber-based material, and a plurality of anti-growth factor antibodies are disposed on the fiber-based material.

In one embodiment of the present invention, the body fluid sample is an aqueous humor and vitreous humor.

In one embodiment of the present invention, the body fluid sample includes an antigen or an antigen fragment capable of being specifically recognized by the anti-growth factor antibodies.

As mentioned above, the detection device, detection strip and the detection system of the present invention is applied with a reaction portion including fiber-based material and anti-growth factor antibodies disposed thereon. The detection device, detection strip and the detection system detect the antigen included in the body fluid of from an organism, such as the angiogenic factors in aqueous humor. It is preferably used for detecting the vascular endothelial growth factor. Since the process of traditional ELISA needs to be done with specific instruments and detection apparatus, the process is thus more complicated. The detection device, detection strip and the detection system of the present invention directly applies the body fluid on the detection device without additional process. Since the substrate of fiber-based material obtains better permeability and water retention ability, which is advantageous for small amount of sample. For users, the addition and detection process are both with intuition. Thus, the detection device of the present invention is further advantageous for easy and fast operation.

### BRIEF DESCRIPTION OF THE DRAWINGS

The present invention will become more fully understood from the subsequent detailed description and accompanying drawings, which are given by way of illustration only, and thus are not limitative of the present invention and wherein:

FIG. 1A shows schematic view of a detection device according to preferred embodiment of the present invention.

FIG. 1B shows partially schematic view of the region A of the detection device according to FIG. 1A.

FIG. 1C shows schematic view of section line A-A shown in FIG. 1A.

FIG. 2A shows schematic view of a detection device according to another preferred embodiment of the present invention.

FIG. 2B shows shows schematic view of section line a-a shown in FIG. 2A.

FIG. 3 shows schematic view of a detection device according to another preferred embodiment of the present invention.

FIG. 4 shows schematic view of detection strip according to preferred embodiment of the present invention.

FIG. 5A shows schematic view of a detection device according to another preferred embodiment of the present invention.

FIG. 5B shows schematic view of a detection device according to another preferred embodiment of the present invention.

FIG. 6A shows the result of the average intensity ratio of vascular endothelial growth factor by paper-based detection device applying ELISA system.

FIG. 6B shows the result of the average intensity ratio of vascular endothelial growth factor by paper-based detection device applying ELISA system.

### DETAILED DESCRIPTION OF THE INVENTION

The present invention will be apparent from the following detailed description, which proceeds with reference to the accompanying drawings, wherein the same references relate to the same elements.

The detection device of the present invention is applied with Enzyme-linked immunosorbent assay (ELISA). The analyte of the present invention is obtained from the body fluid sample of an organism, preferably from the aqueous humor and vitreous humor of the organism. The word “aqueous humor and vitreous humor” used here is collectively referred to the fluid existed in eye cavity; more specifically speaking, referring to the aqueous humor in anterior chamber and the vitreous humor in posterior chamber.

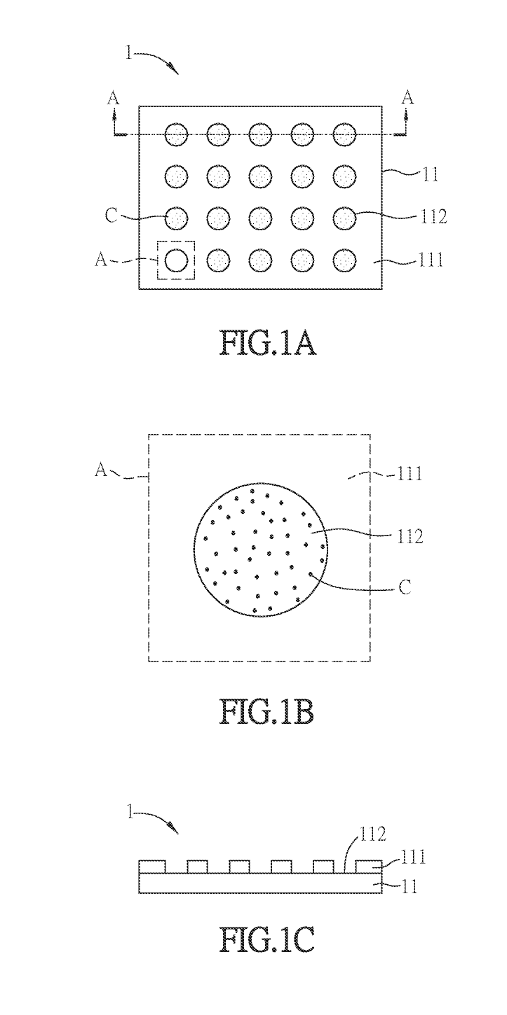

FIG. 1A shows schematic view of a detection device according to preferred embodiment of the present invention, FIG. 1B shows partially schematic view of the region A of the detection device according to FIG. 1A, and FIG. 1C shows schematic view of section line A-A shown in FIG. 1A. With reference to FIG. 1A˜1C, the detection device 1 includes a substrate 11 and a plurality of anti-growth factor antibody C. The substrate 11 includes at least one non-reaction portion 111 and at least one reaction portion 112. This embodiment is based on a substrate 11 having a non-reaction portion 111 as an example. The non-reaction portion 111 defines the plural reaction portions 112. That is, the reaction portions 112 are separated by the non-reaction portion 111. However, the number, shape, size of the reaction portion 112 is not limitation of the present invention. It can be designed according to the need of the experiment.

With reference to FIG. 1A and FIG. 1B, in this embodiment, the non-reaction portion 111 of the substrate 11 is covered by hydrophobic material. Thus, the non-reaction portion 111 is also called hydrophobic region. In detail, the method of forming the hydrophobic non-reaction portion 111 includes wax printing. Wax printing applies device with wax spray function, such as a printer. Wax is disposed on the substrate 11 according to the user's option of figure, shape, or size. With the hydrophobic non-reaction portion 111, the body fluid sample is restricted in the reaction portion 112 in order to prevent the loss of the sample and improve the level of detection accuracy.

However, the formation method of the non-reaction portion 111 is not limitation of the present invention. In practical use, the hydrophilic substrate 11 may alternatively be coated with photoresist layer. Specifically speaking, when the negative resist is applied, such as SU-8 epoxy-based negative photoresist, the region irradiated by UV light would not dissolve in photoresist developer. Thus, the non-reaction portions 111 are formed. On the contrary, the regions which are not irradiated by UV light form the hydrophilic reaction portion 112. However, the detailed forming method is well-understood by the person having ordinary skill in the art, and is not repeated here.

The substrate 11 applies fiber-based material, such as botanical fiber 1 as its material; preferably the material is a high-density fiber. In this embodiment, the high density fiber-based material with an average pore size ranged from 0.7 to 12 micrometers, preferably ranged from 1 to 10 micrometers. The practical application range and preferable application range both includes the combination of any two integers in the above mentioned range. While choosing substrate with larger pore size, the body fluid sample may affected by the action of siphon force and capillary force, thus providing faster flow rates; on the contrary, while choosing substrate with smaller pore size, the flow rate of the body fluid sample may become relatively slower because of the action of siphon force. On the detection effect side, higher flow rate may not be advantageous for the detection target immobilizing in the reaction portion 112, causing the false-negative detection result; on the contrary, more non-specific binding may occurred with lower flow rate, causing the false-positive detection result. Therefore, in practical use, the average pore size of high density fiber-based material is selected according to the component included in the body fluid sample, thus being adjusted by the practical use and need.

In this embodiment, the reaction portion 112 is the region surrounded and defined by the hydrophobic non-reaction portion 111 (i.e. partial region of substrate 11). Thus, the reaction portion 112 also includes the hydrophilic ability the same as the fiber-based material. The reaction portion 112 maintains and absorbs the body fluid by the capillary action generated by the fiber-based material. Furthermore, the superficial body fluid is able to be wicked, diffusing and transferring in the reaction portion due to the density and the minor groove of the fiber-based material. Compared to the nitrocellulose paper applied in the prior technique used for absorbing the sample merely on the surface, the high density fiber-based material of the present invention includes better water permeability, thus providing larger amount of solvent absorption and effectively improving the subsequent detection accuracy of the ELISA analysis.

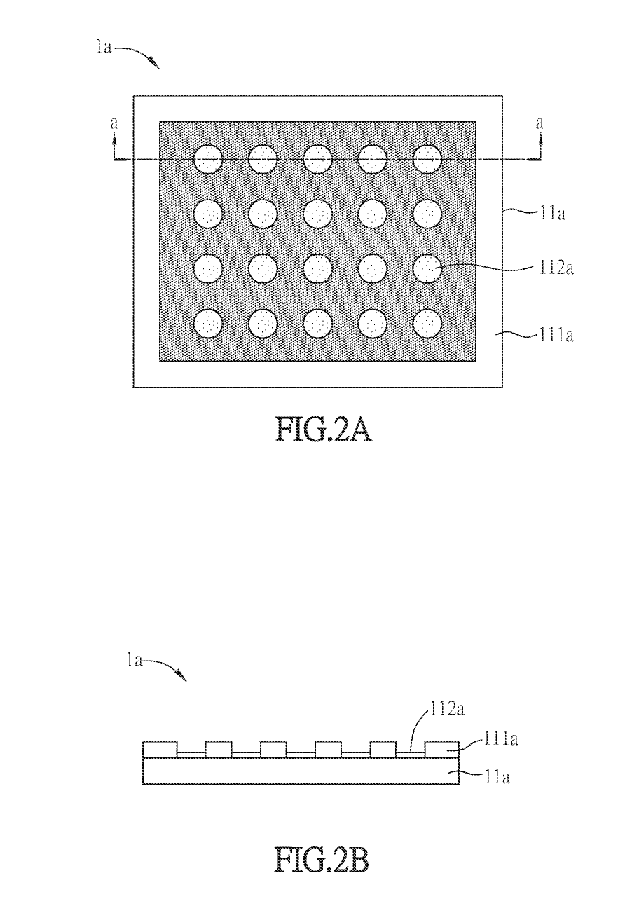

In other embodiments, the reaction portion may disposed by additionally adding fiber-based material on the substrate. With reference to FIG. 2A and FIG. 2B, the same as the above-mentioned embodiment, the reaction portion 112a of the detection device 1a is defined by the non-reaction portion 111a through wax printing. The reaction portion is additionally disposed with fiber-based material in order to play the detection region for superficial body fluid detection. And the material of the substrate 11a is not limited to high density fiber-based material.

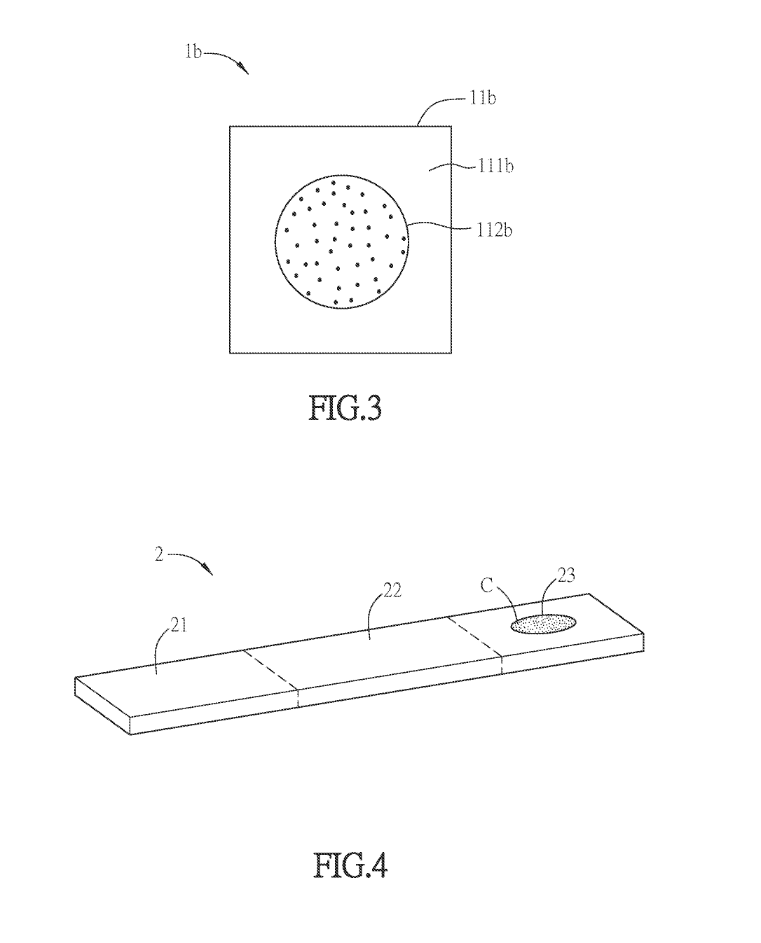

The amount of the reaction portion is not limited. In other embodiment, as shown in FIG. 3, the substrate 11b of the detection device lb may include only one reaction portion 112b and one non-reaction portion 111b, which designed according to the practical detection use.

With reference to FIG. 1B, in this embodiment, the detection device 1 is applied for detecting eye vascular lesion. The detection device 1 is applied for detecting or tracking some common eye vascular lesion, such as retinal vein occlusion (RVO), proliferative diabetic retinopathy (PDR), diabetic macular edema (DME), neovascular glaucoma (NVG) or age-related macular degeneration (AMD). Formation of above-mentioned eye-related diseases is caused by the generation of new blood vessels. In detail, the angiogenic factors release because of hypoxia or other causes during the disease process. These angiogenic factors promote endothelial cell proliferation, further causing microvascular endothelial cells to form a wall gap and resulting in increased permeability, loss of fibrinogen and change of the surrounding connective tissue. This provides environment for new vessels to form. Fluid and blood exudated from the newly formed vessels damage the structure of retina and reduce patients' vision. Angiogenic factors include vascular endothelial growth factor, fibroblast growth factor, platelet-derived endothelial growth factor and angiopoetin.

According to the above, because the aqueous humor and vitreous humor of this kind of patients includes angiogenic factors with quantitative proportion according to the disease activity or severity. Therefore, the detection device 1 applies angiogenic factors (especially VEGF) as a detection target to conduct the subsequent ELISA analysis.

In this embodiment, a plurality of anti-growth factor antibodies C are immobilized in the fiber-based material in reaction portion 112. The anti-growth factor antibodies of the present invention take anti-angiogenic factor antibody for example, which is preferably the vascular endothelial growth factor, and is not for limited sense. The anti-growth factor antibodies C is chosen according to the detection target. Otherwise, the amount of anti-growth factor antibodies C is for exemplary, not for limitation. However, the detailed method of disposing the antibody on the substrate is well-understood by the person having ordinary skill in the art. For example, solution including type XVII collagen C is rinsed in the reaction portion 112, then being dried in order to fix the type XVII collagen C.

After the design of the detection device 1, the detection device 1 is further applied for detecting the amount of endothelial growth factor. In detail, body fluid sample is collected from organism's aqueous humor. At the same time, the body fluid sample is absorbed on the reaction portion 112 of the detection device 1. The fiber-based material of the reaction portion 112 is able to absorb the superficial body fluid of the affected area. The method for collecting aqueous humor includes collecting them from surgical openings in cataract surgery or anterior chamber paracentesis by puncturing. The detection device 1, for example, is for detecting vascular endothelial growth factor in body fluid of organism. Also, the detection device 1 can be applied for other types of growth factors, such as fibroblast growth factor (FGF), platelet-derived endothelial growth factor (PDEGF) or angiopoetin.

When the body fluid includes vascular endothelial growth factors, the anti-growth factor antibody C (the present embodiment takes anti-vascular endothelial growth factors antibody for example) in the reaction portion 112 may interactively react with the antigens. The detection method applying ELISA analysis is not the limitation of the present invention. Its practical detection method is as follows, the anti-growth factor antibodies C are able to specifically recognize the vascular endothelial growth factors. Specific combination may occur between the antibody and the antigen. Then, the extra and uncombined superficial body fluid is washed away. The second antibody with enzyme is added and combined with the vascular endothelial growth factors. Extra and uncombined second antibody is then washed away. Enzyme substrate is then added to make the enzyme show its color to assess whether organisms have vascular endothelial growth factors or not. The colorimetric results may used for estimating the amount of the vascular endothelial growth factors in order to achieve the purpose of qualitative and quantitative test.

The amount of antibodies of vascular endothelial growth factors can be determined by colorimetric reaction, fluorescence, luminescence, radiation or other signals. Specifically speaking, ELISA uses enzymes and reagents to induce colorimetric reaction so as to display presence of the antigens or analyte. Other methods comprising fluorescence, luminescence and real-time PCR reagents generating recognizable signals can also be used. The above mentioned quantitative methods are not limitation of the present invention and could be obtained in the scope of the present invention.

The maximum amount of aqueous humor which can be extracted from the anterior chamber of eyeballs is 0.2 mL, which is not as much as the body fluid amount of blood or urine. Therefore, while using traditional ELISA for VEGF in anterior chamber detection, complicated addition of reagent and wash process often lead to human error. Therefore, the test results cannot be obtained with high confidence. Since the detection device of the present invention can be applied without complicated procedures, thus avoiding human error. Since the substrate of fiber-based material obtains better permeability and water retention ability, which is advantageous for small amount of sample (about 0.04 mL) and solving the difficulty of inadequate specimen volume of aqueous humor. That is, the detection device of the present invention can be applied with small sample volume.

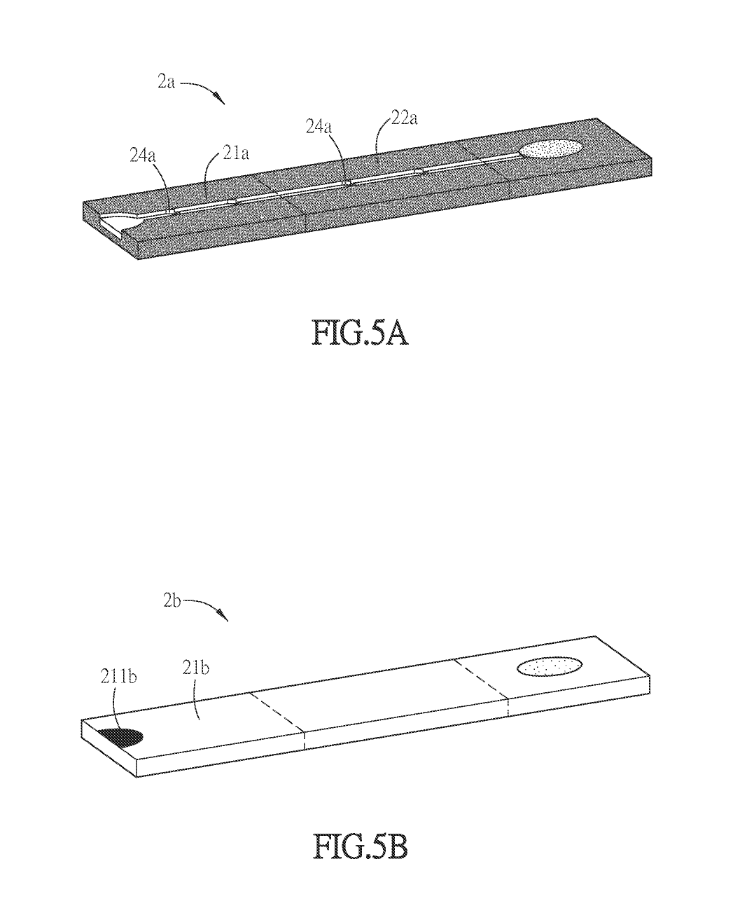

To improve the portability and applicability of the detection device of the present invention, the detection device 1 can be applied a kind of detection strip of the present invention. With reference to FIG. 4, the detection strip 2 includes a sampling portion 21, a transferring portion 22, and a reaction portion 23. The transferring portion is disposed between the sampling portion 21 and the reaction portion 23. The structure of the reaction portion 23 is substantially the same as the reaction portion 13 of detection device 1. The reaction portion 23 includes a fiber-based material, and a plurality of anti-growth factor antibodies C are immobilized on the fiber-based material.

Since the reaction portion 23 of the detection strip 2 needs to be undergone qualitative or quantitative detection, the material of the reaction portion 23 is preferably chosen from high density fiber-based material with an average pore size ranged from 0.7 to 12 micrometers, preferably ranged from 1 to 10 micrometers. The practical application range and preferable application range both includes the combination of any two integers in the above mentioned range. The superficial body fluid transferred to reaction portion can be detected according to the same method as the prior embodiment, and is not repeated here.

In detail, in this embodiment, the sampling portion 21, the transferring portion 22 and the reaction portion of detection strip 2 comprise materials obtaining capillary force ability in order to provide the superficial body fluid sampled by the sampling portion 21 to conduct capillary action. The material used in sampling portion 21 and transferring portion 22 is not the limitation of the present embodiment. The materials can be chosen from cotton fiber, nitrocellulose, glass fiber, or even the same high density fiber-based material as the reaction portion 23 to improve its capillary ability.

The present invention does not limit the way the sampling portion 21 of detection strip 2 used for sampling the body fluid. The detection strip may directly contact the organism or use other assistive devices, such as cotton swabs or needles for sampling and adding to sampling portion 21. Then, body fluid sample can be transferred from transferring portion 22 to reaction portion 23 to conduct the subsequent reaction.

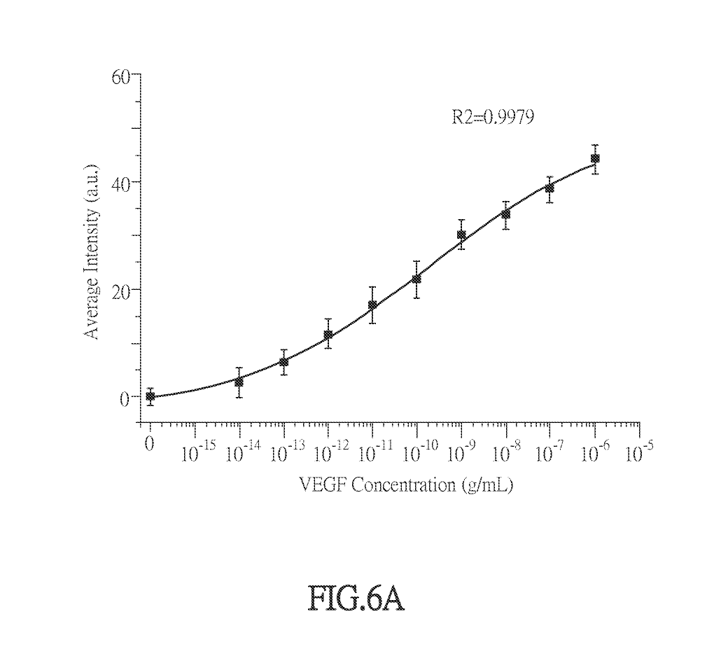

In practical use, the average pore size of the fiber material is chosen according to the analyte and the component in detection reagent. The design of detection strip 2 can be adjusted with other special purposes. In other embodiment, with reference FIG. 5A, the detection strip 2a can be processed with additional hydrophobic treatment. For example, at least part of the sampling portion 21a, the transferring portion 22a, and the reaction portion 23 of the detection strip 2a may be coated with Polydimethylsiloxane (PDMS). With the above process, the hydrophilic area of the detection strip 2a can be defined and minified in order to transfer the sample to the reaction portion 23a through hydrophilic area. Then, the sample is able to react with chemical reagent. With reference to FIG. 5A, the present embodiment further disposes a microchannel structure 24a at sampling portion 21 a or transferring portion 22a to control the flow rate of the body fluid sample and the reaction rate of the same.

Or with reference to FIG. 5B, in order to filter the dander tissue in body fluid sample, a filtering layer 211b may be disposed on the detection strip 2b. However, the shape, size, and the position of the filtering layer 211b is not the limitation of the present invention. The filtering layer is designed according to actual requirement. For example, when a large amount of body fluid sample is provided, the detection strip may include a larger filtering layer to improve the filtering effect.

The present invention further provides a detection system applied for detecting body fluid of an organism. The detection system comprises a detection strip and a monitoring device. The detection strip described herein has substantially the same structure as the detection strip 2 of the prior embodiment. The detection strip of the detection system includes a sampling portion, a transferring portion and a reaction portion, and is not repeated here. The monitoring device detects the interactive reaction of the body fluid sample and the anti-growth factor antibodies for qualitative or quantitative detection. The specific embodiment is conducted with the quantitative method of ELISA analysis. For example, in one embodiment, if the application uses colorimetric enzyme combined with the secondary antibody, the monitoring device may be an instrument capable of receiving optical signals to detect the color reaction of the colorimetric enzyme. Other detection methods, such as the detection of fluorescence, luminescence, and radiation, are well-understood by the person having ordinary skill in the art, and are not repeated here.

The following and accompanying figures take a number of experiments for examples to describe the practical operation method and effect of the detection device and the detailed method of the detection of bullous pemphigoid using the detection device in accordance with the embodiments of the present invention.

### Experiment 1

### Detection of the VEGF by Paper-Based Detection Device Applying ELISA System

According to the present invention, it first provides a chromatography filter paper plate. After moistening the paper plate, VEGF antigens are added thereon and stand for 5 to 7 minutes. Then, bovine serum albumin (BSA) is added as blocking agents to prevent non-specific binding. After standing for 5 to 7 minutes, anti-VEGF antibodies which conjugate HRP are added to react with the antigens for 7 to 10 minutes. Next, the second blocking agent Streptavidin is added to react with reagents for 7 to 10 minutes. Finally, the chromatography filter paper plate is rinsed. Meanwhile, the solution comprising 3, 3′, 5, 5′-tetramethylbenzidine (TMB) and H2O2 are also added thereon until dry. After capturing the images of the paper plate, the images can be analyzed for gaining information.

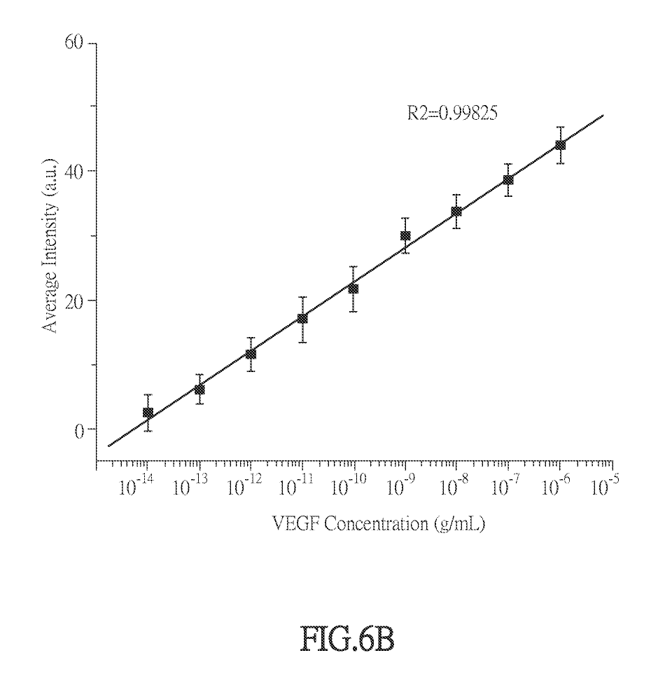

As shown in FIG. 6A and FIG. 6B, they are calibration curves illustrating logarithmic value of the VEGF antigen concentration absorbed in each testing region based on average intensity of colorimetric reaction derived from HRP enzyme reacting in ELISA testing. Each point of the curve is the average value repeated for eight times (N=8) and error bars represents standard deviation of the detected results. The analysis method is first scanning the 96-wells plate, and then analyzing the color strength of each testing region by Photoshop®. By using Hill Equation, the R2 value is 0.9979 matching the data (As shown in FIG. 6A). In addition, the range of the curve between 1.6×10−1˜10−2 (log μg/mL) can be linearly approximated. And the R2 value of the curve which meets the linear curve is 0.99825.

### Experiment 2

### The Detection of VEGF Concentration Produced by Diabetes Patients with Retinopathy Symptom by Paper-Based Detection Device Applying ELISA System

Determined by the above-mentioned method, the average VEGF concentration of the diabetes patients with retinopathy symptom is 740.1±267.7 pg/mL (total samples=14).

### Experiment 3

### The Detection of VEGF Concentration Produced by Age-Related Macular Degeneration Patients by Paper-Based Detection Device Applying ELISA System

Determined by the above-mentioned method, the average VEGF concentration of the age-related macular degeneration patients is 383±155.5 pg/mL (total samples=17).

### Experiment 4

### The Detection of VEGF Concentration Produced by Patients with Retinal Vein Occlusion by Paper-Based Detection Device Applying ELISA System

Determined by the above-mentioned method, the average VEGF concentration of patients with retinal vein occlusion is 219.4±92.1 pg/mL (total samples=10).

As mentioned above, the detection device, detection strip and the detection system of the present invention is applied with a reaction portion including fiber-based material and anti-growth factor antibodies disposed thereon. The detection device, detection strip and the detection system detect the antigen included in the body fluid of from an organism, such as the angiogenic factors in aqueous humor. It is preferably used for detecting the vascular endothelial growth factor. Since the process of traditional ELISA needs to be done with specific instruments and detection apparatus, the process is thus more complicated. The detection device, detection strip and the detection system of the present invention directly applies the body fluid on the detection device without additional process. Since the substrate of fiber-based material obtains better permeability and water retention ability, which is advantageous for small amount of sample. For users, the addition and detection process are both with intuition. Thus, the detection device of the present invention is further advantageous for easy and fast operation.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A detection device applied for detecting a body fluid sample, comprising:
a substrate including at least one reaction portion with a fiber-based material; and
a plurality of anti-growth factor antibodies disposed on the fiber-based material.

2. The detection device according to claim 1, wherein the body fluid sample is an aqueous humor and vitreous humor.

3. The detection device according to claim 1, wherein the substrate includes at least one non-reaction portion, and the non-reaction portion is covered by hydrophobic material.

4. The detection device according to claim 3, wherein the substrate includes two reaction portions, and the reaction portions are separated by the non-reaction portion.

5. The detection device according to claim 1, wherein the fiber-based material is high density fiber-based material with an average pore size ranged from 0.7 to 12 micrometers.

6. The detection device according to claim 1, wherein the anti-angiogenic factor antibodies are anti-angiogenic factor antibody.

7. A detection strip applied for detecting a body fluid sample, comprising:
a sampling portion;
a transferring portion; and
a reaction portion, wherein the transferring portion is disposed between the sampling portion and the reaction portion, the reaction portion includes a fiber-based material, and a plurality of anti-growth factor antibodies are disposed on the fiber-based material.

8. The detection strip according to claim 7, wherein the body fluid sample is an aqueous humor and vitreous humor.

9. The detection strip according to claim 7, wherein the fiber-based material is high density fiber-based material with an average pore size ranged from 0.7 to 12 micrometers.

10. The detection strip according to claim 7, wherein the anti-growth factor antibodies are anti-angiogenic factor antibodies.

11. A detection system applied for detecting a body fluid sample of an organism, comprising:
a detection strip comprising a sampling portion, a transferring portion and a reaction portion, the transferring portion is disposed between the sampling portion and the reaction portion, the reaction portion includes a fiber-based material, and a plurality of anti-growth factor antibodies are disposed on the fiber-based material; and
a monitoring device, the monitoring device detects the interactive reaction of the body fluid sample and the anti-growth factor antibodies.

12. The detection system according to claim 11, wherein the body fluid sample is an aqueous humor and vitreous humor.

13. The detection system according to claim 11, wherein the body fluid sample includes an antigen or an antigen fragment capable of being specifically recognized by the anti-growth factor antibodies.
